(19) United States
(12) Patent Application Publication
(10) Pub. No.: US 2024/0197404 A1
(43) Pub. Date: Jun. 20, 2024

(54) Method and Apparatus for Imaging a Subject

(71) Applicant: Medtronic Navigation, Inc., Louisville, CO (US)

(72) Inventor: Saba PASHA, Houston, TX (US)

(21) Appl. No.: 18/066,640

(22) Filed: Dec. 15, 2022

(51) Int. Cl. A61B 34/20; A61B 8/00; A61B 8/08

(52) U.S. Cl. CPC A61B 34/20; A61B 8/0841; A61B 8/4209; A61B 8/4245; A61B 8/4477; A61B 8/461; A61B 8/54; A61B 8/56; A61B 2034/2051; A61B 2034/2063

(57) ABSTRACT

Disclosed is a system to plan and position an implant in a subject. The planned position may be based upon various features and structures identified in a group of subjects for a current subject. The implant may then be positioned in a selected position including a relative position and orientation of one or more electrodes on the implant which may be identified as an optimal position for the selected current subject.

### FIELD

The present disclosure is related to a system for assisting in a procedure on a subject, such as imaging a subject and tracking an imaging device and/or an instrument.

### BACKGROUND

This section provides background information related to the present disclosure which is not necessarily prior art.

A subject, such as a human patient, may undergo a procedure. The procedure may include a surgical procedure to correct or augment an anatomy of the subject. The augmentation of the anatomy can include various procedures, such as movement or augmentation of bone, insertion of an implant (i.e., an implantable device), or other appropriate procedures.

A surgeon can perform the procedure on the subject with images of the subject that are based on projections of the subject. The images may be generated with one or more imaging systems such as a magnetic resonance imaging (MRI) system, a computed tomography (CT) system, or a fluoroscopy (e.g., C-Arm imaging systems).

### SUMMARY

This section provides a general summary of the disclosure and is not a comprehensive disclosure of its full scope or all of its features.

A portion of a subject, such as a portion of a human anatomy, can be imaged to generate image data thereof that may be analyzed as data and/or displayed to be viewed by a user. It is understood, however, that a subject may include a non-human subject and/or non-living subject. Non-living subjects may include enclosed structures such as wings, robotic systems, etc. In analyzing the image data, various features may be identified and/or used to identify various additional features. For example, an anatomical landmark may be identifiable in a selected image data

The subject disclosure relates to imaging a subject with one or more ultrasound transducers. The one or more ultrasound transducers may be positioned relative to a subject to image a plurality of portions of the subject. The ultrasound transducers may each be operated individually and/or in concert to obtain image data of the subject. The image data may be used to generate an image of the subject.

The subject may include any appropriate subject, such as a human subject, other living subject, or nonliving subject. The ultrasound transducer may be used to image any appropriate portion of the subject. An image may be generated based upon the ultrasound image data to generate an image of an interior portion of the subject.

One or more ultrasound transducers may be tracked individually or as a group or unit to assist in determining a pose of the ultrasound transducers. Further the subject may be tracked and an instrument relative to the subject and/or the ultrasound transducers may also be tracked. This tracking may assist in selecting which one or sub-plurality of the plurality of ultrasound transducers to acquire image data from the subject.

A system, such as a navigation system, may tract one or more of the ultrasound transducers, the instrument, and the subject. The navigation system may generate an image based upon image data received from one or more of the ultrasound transducers. In various embodiments, for example, an image may be generated based upon image data received or acquired from only one of the ultrasound transducers. The single ultrasound transducer may be selected based upon a tract or known position of the instrument relative to the subject and/or the ultrasound transducer.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is an environmental view of an operating suite;

FIG. 2 is a schematic view of the imaging system positioned relative to a subject, according to various embodiments;

FIG. 3A is a schematic view of the imaging system positioned relative to a subject, according to various embodiments;

FIG. 3B is a schematic view of the imaging system of FIG. 3A positioned relative to a subject, according to various embodiments;

FIG. 4A is a schematic view of the imaging system positioned relative to a subject, according to various embodiments;

FIG. 4B is a schematic view of the imaging system of FIG. 4A positioned relative to a subject, according to various embodiments;

FIG. 5 is a flowchart of an operation of the imaging system, according to various embodiments;

FIG. 6A is a schematic view of the imaging system positioned relative to a subject, according to various embodiments;

FIG. 6B is a schematic view of the imaging system of FIG. 6A positioned relative to a subject, according to various embodiments;

FIG. 7 is a flowchart of an operation of the imaging system, according to various embodiments;

FIG. 8A is a schematic view of the imaging system positioned relative to a subject, according to various embodiments; and

FIG. 8B is a schematic view of the imaging system of FIG. 8A positioned relative to a subject, according to various embodiments.

Corresponding reference numerals indicate corresponding parts throughout the several views of the drawings.

### DETAILED DESCRIPTION

Example embodiments will now be described more fully with reference to the accompanying drawings.

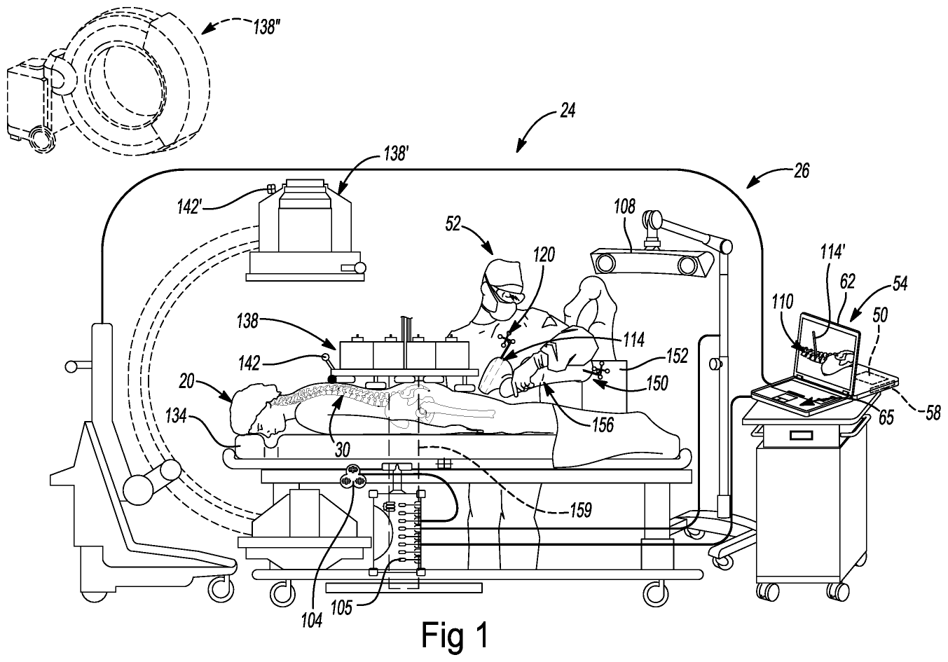

With initial reference to FIG. 1, a subject 20 may be any appropriate subject. Although the following discussion relates to a human subject, it is understood that any appropriate living or non-living subject may be provided or be within the scope of the subject disclosure. For example, a non-human living subject may be evaluated and a selected procedure performed thereon. Further, various non-living subjects may have image data acquired of internal portions and a procedure may be determined, planned, and performed within an outer housing or body (such as a hull) of the non-living subject. Various non-living subjects include internal portions of motors, hulls, or other appropriate subjects. Also, while the following discussion refers exemplarily to performing a procedure relative to a spine of a human subject 20, other appropriate implants and/or therapies are within the scope of the subject disclosure.

The subject 20 may be positioned in a suite 24 for a selected procedure. The suite may include various systems, such as a navigation system 26. The navigation system 26 may include various portions such as a selected processor module 50 that accesses selected memory and or input from a user 52. The processor module 50 may be a general-purpose processor and/or an application specific processor module (e.g., application specific integrated circuit (ASIC)). The processor module 50 may be included in and/or accessed by an exemplary processor system 54 that may include the processor module 50 and a memory module 58. An output may also be made and may include a display device 62. The navigation system may further include one or more inputs 65, such as a keyboard, a touch pad, a touch screen, a mouse, etc.

According to various embodiments, the navigation system 26 may include a surgical navigation system including those sold by Medtronic Navigation, Inc., such as the Stealth Station® surgical navigation system. Briefly, in surgical navigation, the subject 20 may be tracked with a selected tracking device, such as a subject tracking device 100. The subject tracking device 100 may be associated, such as fixed, to a portion of the subject 20 such as on and/or relative to a spine 30 of the subject 20. The subject tracking device 100 may be tracked with an appropriate tracking system such as an electromagnetic tracking system 104 and/or an optical tracking system 108. It is understood that other appropriate tracking systems may be used and the EM 104 and optical 108 tracking systems are merely exemplary.

The tracking systems may track the position of the patient tracker 100 and maintain a registration of a patient space to another selected space, such as an image space. The tracking system and/or the navigation system 26 may track and determine a relative pose of the patient tracking device 100 to an image displayed on the display 62, such as the image 110. A device, also referred to as an instrument, may also be tracked. An instrument 114 may be tracked with an instrument tracker 120. A device representation, such as a graphical representation 114′ thereof, may be displayed on the display device 62 relative to the subject image 110 based upon a tracked location of the device 114. The device tracking device or device tracker 120 may be tracked with the tracking system 104, 108, as is understood by one skilled in the art. It is also understood by one skilled in the art that the tracked position and navigated position of the device 114 may be performed based upon a registration of the image or image space 110 to the subject or subject space of the subject 20. As discussed herein, various registrations may also occur between the subject image 110 and/or additional imaged portions such as structures in various different image modalities.

The positioning of the device 114 may be performed in the selected suite 24, such as a surgical suite. The surgical suite 24 may include selected structures or portions such as a patient support 134 and an imaging system 138. The imaging system 138 may be used to generate or acquire image data of the subject 20, according to various embodiments. The imaging system 138 may also be tracked with an imaging system tracker 142, as is generally understood by one skilled in the art. The imaging system 138, as discussed herein, may include one or more ultrasound transducers for obtaining image data of the subject 20. Additional and/or alternative imaging systems may include a C-arm x-ray imaging 138′ system and/or an O-arm® imaging system 138″, sold by Medtronic, Inc. The image data of the subject 20 may be acquired with any appropriate imaging system, such as the imaging systems 138, 138′, 138″ at any appropriate time such as prior to the procedure, during the procedure, and/or after the procedure. Further, the tracking systems and/or the various tracking devices may be incorporated into a surgical navigation system, according to various embodiments.

The additional and/or alternative imaging system 138″ can include an O-Arm® imaging system sold by Medtronic Navigation, Inc. having a place of business in Louisville, CO, USA. The imaging system 138″, including the O-Arm® imaging system, or other appropriate imaging systems may be in use during a selected procedure, such as the imaging system described in in U.S. Pat. Nos. 7,188,998; 7,108,421; 7,106,825; 7,001,045; 6,940,941; 11,344,268; 11,399,784; and U.S. patent application Ser. No. 13/016,718 published on Apr. 26, 2012 as U.S. Pat. App. Pub. No. 2012/0099768, all of the above incorporated herein by reference. Further, the imaging system may include various features and elements, such as a slotted filter, such as that disclosed in U.S. Pat. Nos. 10,881,371 and 11,071,507 to Helm et al., all of the above incorporated herein by reference. Other appropriate imaging systems may include C-arm imaging systems including an opposed x-ray source and x-ray detector and related processor modules and/or memory.

The various tracking devices 100, 120, 142, 142′ can be tracked with the navigation system including one or more of the tracking systems 104, 108 and the information can be used to allow for displaying on the display 62 a pose of an item, e.g., the tool or instrument 114. For example, the instrument graphical representation 114′ may be displayed alone and/or superimposed on any appropriate image, such as the image of the subject 110. The instrument 114 may be operated, controlled, and/or held by the user 52. The user 52 may be one or more of a surgeon, nurse, welder, etc. Briefly, tracking devices, such as the patient tracking device 100, the imaging device tracking device 142, and the instrument tracking device 120, allow selected portions of the operating theater 24 to be tracked relative to one another with the appropriate tracking system, including the optical localizer 108 and/or the EM localizer 104. It is understood, however, that other tracking modalities may be used such as ultrasound, acoustic, radar, etc. Generally, tracking occurs within a selected reference frame, such as within a patient reference frame.

It will be understood that any of the tracking devices 100, 120, 142 can each be optical, EM tracking devices, or other appropriate tracing device and/or more than one type of tracking device depending upon the tracking localizer used to track the respective tracking devices. It is understood that the tracking devices 100, 120, 142 may all be similar or different and may all be interchangeable but selected or assigned selected purposes during a navigated procedure. It will be further understood that any appropriate, such as alternative or in addition thereto, tracking system can be used with the navigation system. Alterative tracking systems can include radar tracking systems, acoustic tracking systems, ultrasound tracking systems, and the like.

An exemplarily EM tracking system can include the STEALTHSTATION® AXIEM™ Navigation System, sold by Medtronic Navigation, Inc. having a place of business in Louisville, Colorado. Exemplary tracking systems are also disclosed in U.S. Pat. No. 7,751,865, issued Jul. 6, 2010; U.S. Pat. No. 5,913,820, issued Jun. 22, 1999; and U.S. Pat. No. 5,592,939, issued Jan. 14, 1997, all incorporated herein by reference.

Further, regarding EM tracking systems it may be necessary to provide shielding or distortion compensation systems to shield or compensate for distortions in the EM field generated by the EM localizer 104. Exemplary shielding systems include those in U.S. Pat. No. 7,797,032, issued Sep. 14, 2010 and U.S. Pat. No. 6,747,539, issued Jun. 8, 2004; distortion compensation systems can include those disclosed in U.S. patent application Ser. No. 10/649,214, filed on Jan. 9, 2004, published as U.S. Pat. App. Pub. No. 2004/0116803, all of which are incorporated herein by reference.

With an EM tracking system, the localizer 104 and the various tracking devices can communicate through an EM controller 105. The EM controller can include various amplifiers, filters, electrical isolation, and other systems. The EM controller 105 can also control the coils of the localizer 104 to either emit or receive an EM field for tracking. A wireless communications channel, however, such as that disclosed in U.S. Pat. No. 6,474,341, issued Nov. 5, 2002, herein incorporated by reference, can be used as opposed to being coupled directly to the EM controller 105.

It will be understood that the tracking system may also be or include any appropriate tracking system, including a STEALTHSTATION® TRIA®, TREON®, and/or S7™ Navigation System having an optical localizer, similar to the optical localizer 108, sold by Medtronic Navigation, Inc. having a place of business in Louisville, Colorado. Further alternative tracking systems are disclosed in U.S. Pat. No. 5,983,126, issued Nov. 9, 1999, which is hereby incorporated by reference. Other tracking systems include an acoustic, radiation, radar, etc. tracking or navigation systems.

Physical space of and/or relative to the subject, such as the patient 20, may be referred to as subject or patient space. Image space is defined by an image or coordinate system of an image that is generated or reconstructed with the image data from an imaging system, such as the imaging system 138, 138′, 138″ and may be referred to as image space. The image space can be registered to the patient space by identifying matching points or fiducial points in the patient space and related or identical points in the image space. The imaging device 138, 138′, 138″ can be used to generate image data at a precise and known position. This can allow image data that is automatically or “inherently registered” to the patient 20 upon acquisition of the image data. Essentially, the position of the patient 20 is known precisely relative to the imaging system 138, 138′, 138″ due to the accurate positioning of the imaging system 138, 138′, 138″ in the patient space. This allows points in the image data to be known relative to points of the patient 20 because of the known precise location of the imaging system 138, 138′, 138″. It is understood, likewise, that the imaging system may be used to generate image data of the subject 20 at any appropriate time. Further, the imaging system may include one or more of a Magnetic Resonance Imaging (MRI) device, computer tomography (CT) device, etc.

Alternatively, and/or additionally, manual or automatic registration can occur by matching fiducial points in image data with fiducial points on the patient 20. For example, selected patient anatomy (e.g., ear portions, nose tip, brow line, etc.) may be identified in Registration of image space to patient space allows for the generation of a translation map between the patient space and the image space. According to various embodiments, registration can occur by determining points that are substantially identical in the image space and the patient space. The identical points can include anatomical fiducial points or implanted fiducial points. Exemplary tracking and navigation systems and appropriate registration techniques are disclosed in at least one of U.S. Pat. No. 9,737,235 issued Aug. 22, 2017; U.S. Pat. No. 7,751,865 issued Jul. 6, 2010; U.S. Pat. No. 6,474,341 issued Nov. 5, 2002, U.S. Pat. No. 5,913,820 issued Jun. 22, 1999; U.S. Pat. No. 5,592,939 issued Jan. 14, 1997; and/or U.S. Pat. No. 5,983,126 issued Nov. 9, 1999; all of which are incorporated herein by reference.

Once registration has occurred, the navigation system including the tracking systems 104, 108, with and/or including the imaging system 138, 138′, 138″ can be used during performance of selected procedures. Selected procedures can use the image data generated or acquired with the imaging system 138, 138′, 138″ and the tracked pose of one or more tracked items can be displayed relative to the image, such as superimposed thereon. The pose that is determined generally includes a selected number of degrees of freedom, such as six degrees of freedom. These may include at least three degrees of location (x, y, and z-axis locations) and orientation (yaw, pitch, and roll). Further, the imaging system 138, 138′, 138″ can be used to acquire image data at different times relative to a procedure. As discussed herein, image data can be acquired of the patient 20 subsequent to a selected portion of a procedure for various purposes, including confirmation of the portion of the procedure.

Given the navigation system 26 and the various imaging systems 138, 138′, 138″, the following disclosure relates to acquiring image data and displaying on the display 62 the image 110. Various instruments may be tracked relative to the subject 20 and may also be displayed on the display 62, such as with the graphical representation 114′. It is understood, however, that the various systems need not require display of the image 110 and may be used for performing a portion of a procedure using the image data. For example, a robotic system, such as a robotic arm 150 may be positioned relative to the subject 20. The robotic system 150 may include one or more of the Mazor® and/or Mazor X® Spinal Robotics System. The robotic arm 150 may include various portions such as a base 152 and an articulated arm portion 156. The arm portion 156 may include an end effector 160. The end effective 160 may be moved relative to the subject 20, such as the spine 30, to assist in performing a procedure relative to the subject 20. For example, the end effector 160 may be a guide for an instrument such as the instrument 104 as noted above. Therefore, the instrument 114 may be positioned relative to the end effector 160 to perform a procedure on the subject 20. The pose of the end effector 160, therefore, may also be tracked and/or navigated relative to the spine or other portion of the subject 20. The display 62 may display a graphical representation of the end effector 160 and the navigation system 26 may know or determine that the pose of the end effector 160 relative to the subject 20, including a portion thereof such as the spine 30, to assist in performing the procedure.

Further as discussed herein, the imaging system 138 may be the main imaging system discussed. It is understood, however, that any of the other appropriate imaging systems may also be used to acquire image data of the subject 20. In various embodiments, however, the imaging system 138 may include one or more ultrasound transducers 170. Discussion herein to one ultrasound transducer may refer to a plurality of ultrasound transducers being operated separately and/or together, unless specifically indicated otherwise. Further, the imaging system 138 may be used in a robotic guided procedure, minimally or low invasive procedure, or other appropriate procedure on the subject 20. Such procedures may include spinal fusions, disk replacements, prosthesis implantation, etc.

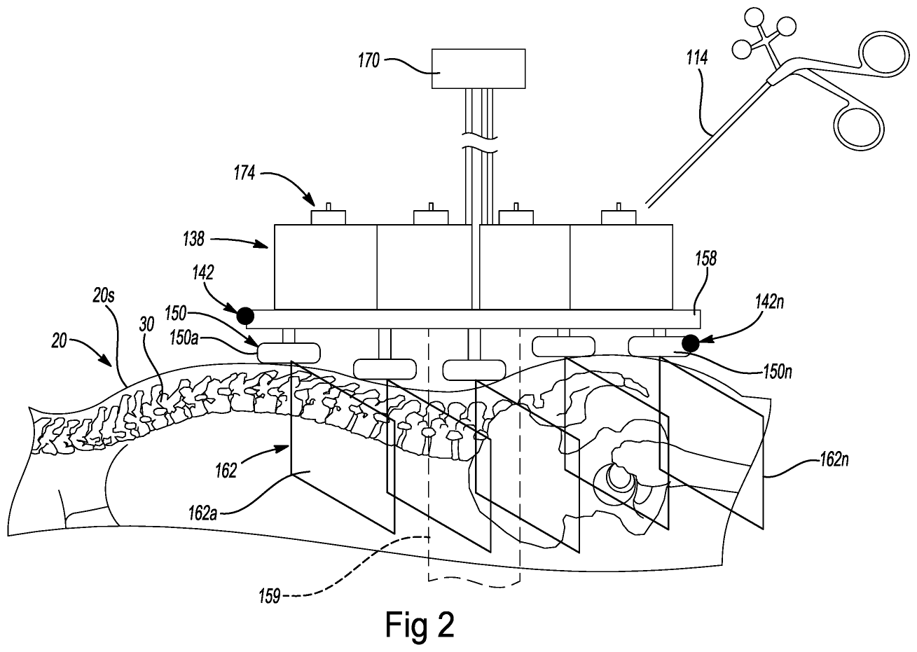

With continuing reference to FIG. 1, and additional reference to FIG. 2, the imaging system 138 will be described in greater detail. The imaging system 138 may be positioned relative to the subject 20. For example, the imaging system 138 may be positioned to acquire image data of the spine 30 of the subject 20. It is understood, however, the imaging system 130 may acquire image data of any appropriate portion of either a human patient, a non-human patient, or any appropriate subject. Therefore, discussion of the spine 30 relative to a human patient at the subject 20 herein is merely exemplary. Further, image data may be acquired of any appropriate portion of any appropriate subject (e.g., heart, brain, femur) and the device 114 may be tracked relative thereto.

The imaging system 138 may include a plurality of imaging elements including one or more ultrasound (US) transducers. The ultrasound transducers may include a selected number of ultrasound transducers and herein may be referred to as a group of ultrasound transducers 150 and/or individually as one of the ultrasound transducers referenced by 150 followed by a letter. The number of US transducers 150 may be selected for any appropriate reason, such as imaging an area or volume to be imaged, size of the subject, cost of the imaging system 138, or other appropriate purposes. The discussion herein of the US transducers 150 may include an initial or first ultrasound transducer 150a and a final ultrasound transducer 150n. The US transducer 150n may refer to any final or selected one of the US transducers and intended to refer that the imaging system 138 may include any appropriate number of the US transducers 150.

The imaging system 138 may be positioned relative to the subject 20 with any appropriate system. For example, a holder or mounting assembly or portion 158 may be positioned relative to the subject 20. The holder 158 may have all of the ultrasound transducers 150 fixed relative thereto such that they may be positioned relative to the subject 20 as a unit, herein referred to as the imaging system 138. The holder 158, according to various embodiments, may be mounted to a mount or arm 159. The mount 159 may be positioned relative to the subject 20. In various embodiments, the mount 159 may be a stand that is positioned on a floor relative to the subject 20. The mount 159 may also or alternatively be connected to the patient support 134. In various embodiments, however, the mount 159 allows the holder 158 to be fixed relative to the subject 20. The user 52, however, may selectively move the mount 159, the holder 148, and/or the ultrasound transducers 150 relative to each other and/or relative to the subject 20. The tracker 142 may be used to maintain registration even if any of these are moved after an initial registration to the subject 20, as is understood by one skilled in the art.

The ultrasound transducers 150 may be fixed relative to the holder 148 and/or movable relative to the holder 158. In various embodiments, the ultrasound transducers 150 may be fixed relative to the holder 158 such that the imaging system 138 is positioned relative to the subject 20 in substantially a similar configuration during each use. It is also understood that the ultrasound transducers 150 may be movable relative to the holder 158 to allow the ultrasound transducers 150 to be moved relative to the holder 158 for selected uses. Nevertheless, at a selected time each of the ultrasound transducers 150 may be held fixed relative to the holder 158 for various purposes, such as tracking the imaging system 138, generating image data of the subject 20, or other purposes.

As noted above, the imaging system 138 may be tracked with the tracker 142. The imaging system tracker 142 may be fixed relative to the holder 158. Therefore, the tracking of the image system tracker 142 allows for tracking a position of the ultrasound transducers 150 fixed relative thereto. A pose of each of the ultrasound transducers 150 relative to the holder 158 may be known. Thus, tracking the imaging system tracker 142 allows for determination of determining a pose of each of the ultrasound transducers 150.

As noted above, the ultrasound transducers 150 may be fixed relative to the holder 158, therefore, a predetermined or pre-known pose of each of the ultrasound transducers 150 relative to the holder 158 may be known. If the ultrasound transducers 150 are immovable relatives of the holder 158, a pose of each of the ultrasound transducers 150 may be input to the respective navigation system 26 or measured with appropriate sensors. For example, position sensors may be used to determine a position of a mounting portion of the ultrasound transducers 150 relative to the holder 158. Regardless, the pose of the ultrasound transducer relative to the holder 158 may be determined.

In addition and/or alternatively, each of the ultrasound transducers 150 may be individually tracked with a selected image system tracker such that each of the ultrasound transducers 150 include an individual tracker. Therefore, for example, the ultrasound transducer 150a may have a tracker 142a connected thereto. Each of the ultrasound transducers may have a tracker connected thereto to allow the individual ultrasound transducers to be tracked during a selected procedure. Therefore, the imaging system 138 may be tracked as a unit and/or each of the individual transducers 150 may be tracked individually with selected individual trackers 142n.

The imaging system 138 may include one or more of the ultrasound transducers 150 that may image of the subject 20. As is understood by one skilled in the art, each of the ultrasound transducers may generate an imaging plane or plane. The imaging plane or space of each US transducer 150 may be referred to as a transducer space, as discussed further herein. Therefore, each of the ultrasound transducers may include a respective ultrasound imaging plane or space 162. Again, as noted herein, each of the ultrasound transducers may generate a plane, therefore, the planes may be referred to together as the planes 162 and/or individually 162a through 162n. The imaging planes 162 of the respective ultrasound transducers 150 may be operated in a selected manner, such as discussed further herein. The ultrasound transducers 150 may be operated together and/or individually based upon selected purposes and imaging, as discussed herein. Therefore, the imaging system 138 may generate an image of the subject 20 that may include a length or span that is longer than an individual ultrasound transducer without requiring movement of any of the ultrasound transducers 150 of the imaging system 138.

Each of the ultrasound transducers 150 may communicate with an imaging system processor 172. The imaging system processor 172 may be a separate processor and/or incorporated with the processor 50 of the navigation system 26. Nevertheless, of the imaging system processor 170 may allow for operation and/or receiving of image data from each of the ultrasound transducers 150 of the imaging system 130. The processor module 170 may operate as a multiplexer of the US transducers 150 of the imaging system 138. In various embodiments, the plurality of US transducers 150 may be multiplexed in an automatic and/or manual manner to operate only selected one or more of the US transducers 150 to generate image data of the subject.

In various embodiments, for example, a switch 174 may be provided for each of the ultrasound transducers 150. The switch may be a mechanical switch and/or an electronic switch. Further, the switch may be separate from and/or incorporated within the imaging processor 170. In various embodiments, the switch may be an operational instruction for any of the selected US transducers 150.

The switch 174 may allow for each of the individual ultrasound transducers 150 to be separately or individually operated. This may reduce crosstalk, interference, and the like between ultrasound transducers if operated simultaneously near one another. Further, as discussed further herein, the switch 174 may allow for individually operating the ultrasound transducers 150 to allow for imaging of a selected portion of the subject 20 based upon a tracked pose of one or more of the ultrasound transducers 150, the imaging system 138, or other portion, such as the tracked device 114. The multiplexing of the US transducers 150 may be automatic, manual, or a combination thereof, as discussed herein.

A communication or a connection between the ultrasound transducers 150 and the processor 170 may be any appropriate connection. For example, a wired connection may be provided between each of the ultrasound transducers 150 and the processor 170. Additionally or alternatively, a wireless connection may be provided between each of the transducers 150 and the processor 170. Thus, the communication for operation and receiving image data from each the ultrasound transducers 150 may be provided in any appropriate manner between the ultrasound transducers 150 and the processor 170.

A selected one or more of the US transducers may be operated to selectively acquire image data based on tracked poses of the device 114. In operation, for various purposes, the device 114 including the device tracker 120 may be tracked with one or more of the localizer systems including the EM localizer 104, the optical localizer 108, or other appropriate localizer. The localizer may be used to track or determine pose information of the tracking device 120 associated with the device 114. A pose of the device 114, including at least a portion thereof, may then be determined based upon the tracked pose of the tracking device 120. Similarly, the transducers 150 may be tracked with of the related tracking device 142. The relative position of the transducers 150 of the imaging system 138 relative to the device 120 may then be determined. The determination may be based upon the tracked pose of both of the device tracking device 120 and the imaging tracking device 142. The determination may be made by the navigation system, including the navigation processor 50, to determine the relative pose of the device 114 and one or more of the transducers 150. Based upon the relative pose of the device 114 and one or more of the transducers 150 a selected one or more of the ultrasound transducers 150 may be operated to acquire image data of the subject 20. For example, as discussed above, a plurality of the US transducers 150 may be positioned relative to the subject 20 to acquire image data relative to each of the respective ultrasound transducers 150. As noted above, one or more of the ultrasound transducers may be operated substantially independently to acquire substantially real time data when operated.

The imaging system 130 may be operated in the various manners to image the subject 20 during a procedure. For example, the instrument or device 114 may be moved relative to the subject 20 to perform or assist in performing a procedure on the subject 20. The imaging system 138 may be used to image the subject 20 relative and the device 114, or at least a portion of the device 114. The device 114 may also include various implants, such as a spinal implant, screw, or the like. Thus, the imaging system 138 may be operated to image the volume or region of the subject 20 where the operation is currently occurring. Thus, real-time or current switching between the various ultrasound transducers 150a-150n of the plurality of ultrasound transducers 150 may be selected and performed. The switching may allow only a single one or few of the ultrasound transducers 150 to operate to a reduce crosstalk and interference between the ultrasound transducers 150. However, including the plurality of the ultrasound transducers 150 with the imaging system 138 may reduce the amount of movement or continuous monitoring of positioning of the ultrasound transducers while attempting to acquire real time image data relative to the device 114 within the subject 20.

Accordingly, the ultrasound transducers 150 may be positioned relative to the subject 20 as the imaging system 138. The imaging system 138 may, therefore, be operated to ensure that only a selected one or selected number of the ultrasound transducers 150 are operating to acquire image data relative to the subject 20. According to various embodiments, including those disclosed herein, the switching and/or operation may be manual, automatic, or combinations thereof.

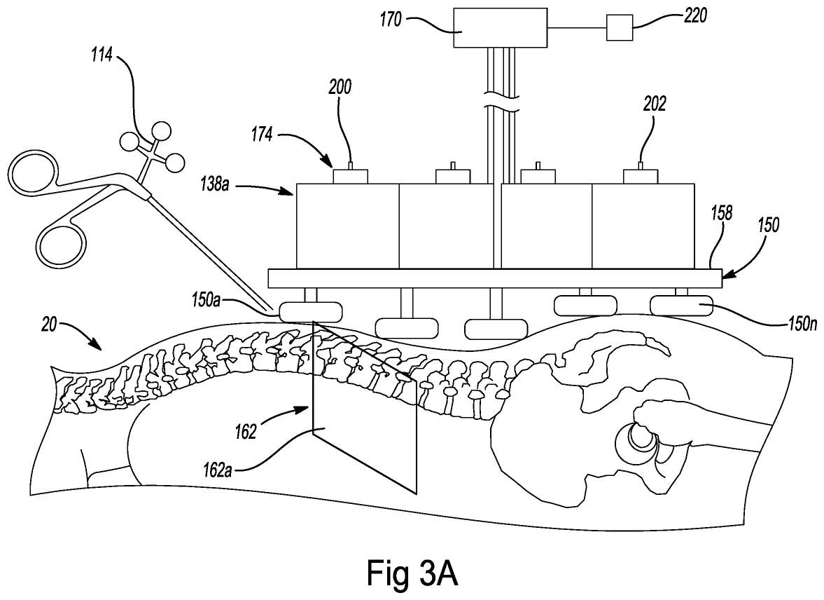

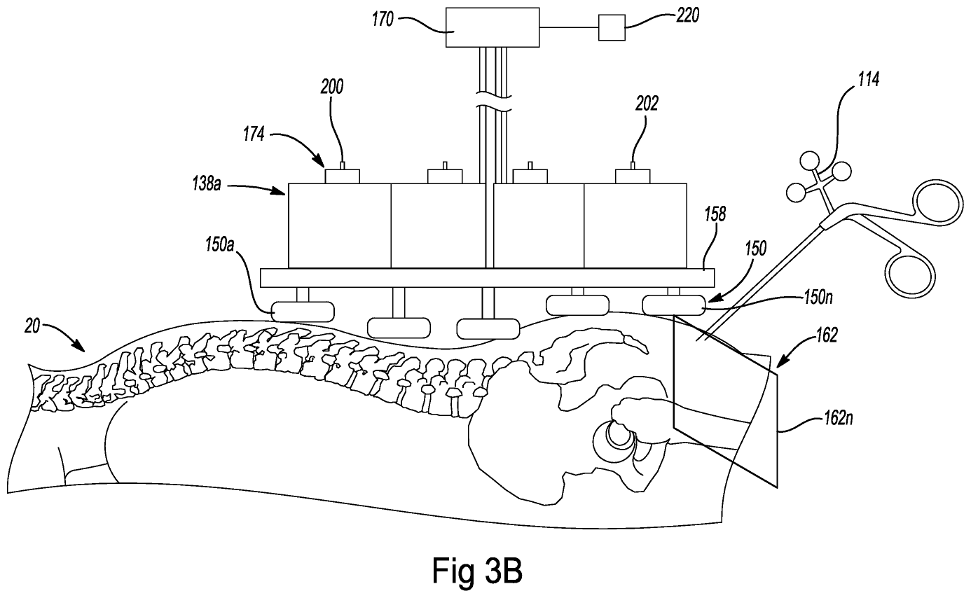

For example, as illustrated in FIG. 3A, the imaging system 138, according to various embodiments, is illustrated as an imaging system 138a and may be positioned relative to the subject 20. The imaging system 138a may be substantially similar to that noted above, with the variations discussed below. The US transducers 150 may be positioned relative to the holder 158 of the imaging system 138a. Each one of the ultrasound transducers 150 may then be operated or selected manually by the user 52. As discussed herein, operation may include selecting for acquisition of image data.

For example, if the device 114 is moved relative to the patient 20 near the first ultrasound transducer 150a, the user 52 may manually turn on or operate the ultrasound transducer of 150a. In various embodiments, for example, the switch 174 may be a manual switch 174m. The manual switch 174m may include a toggle portion 200 such that the user 52 may toggle between an on and off position. The US transducer 150a may then acquire image data in the single transducer space 162a. Similarly, if the device 114 is moved away from the US transducer 150a the user may toggle the toggle switch 200 to the off position.

In a similar manner, as illustrated in FIG. 3B, if the device 114 is moved relative to the ultrasound transducer 150n, the user 52 may toggle a toggle switch portion 202 to turn on the ultrasound transducer 150n. Thus, image data is collected with the US transducers 150n in the plane 162n. Thus, only selected ones of the US transducers are operated at a selected time.

In this manner, only the US transducers 150a-150n that is/are near the position of the device 114 may be operated to generate image data. In various embodiments, the instrument 114 is near one or more of the selected US transducers 150 is the US transducer may image the instrument 114 and/or the portion being affecting by the instrument 114. The image data generated with the operating ultrasound transducer may be used to generate the image 110 that is displayed on the display device 62. The image on the display device 62, therefore, may be based upon a real-time selection of one or more of the ultrasound transducers 150 that may be manually made by the user 52.

While the user 52 may manually operate or turn on one or more of the ultrasound transducers 150, it is understood that the manual switching operation may not be with a manual toggle switch. For example, the input 65 of the processing system 54 may be used to operate, such as turning on and off the ultrasound transducers 150. Similarly or alternatively, an input 220 may be connected with the ultrasound transducers 150, such as through or with the processor system 170. The user 52 may, for example, use a screen, a pedal, or the like to selectively operate one or more of the ultrasound transducers 150. Thus, the user 52 may manually select which of the ultrasound transducers are operated based upon a selection by the user 52 during the procedure.

It is understood that the individual moving the device 114 may be the same user that operates the ultrasound transducers and/or may be a different user. For example, a surgeon may move the device 114 and provide verbal instructions to an assistant to turn on or off selected one or more of the ultrasound transducers 150.

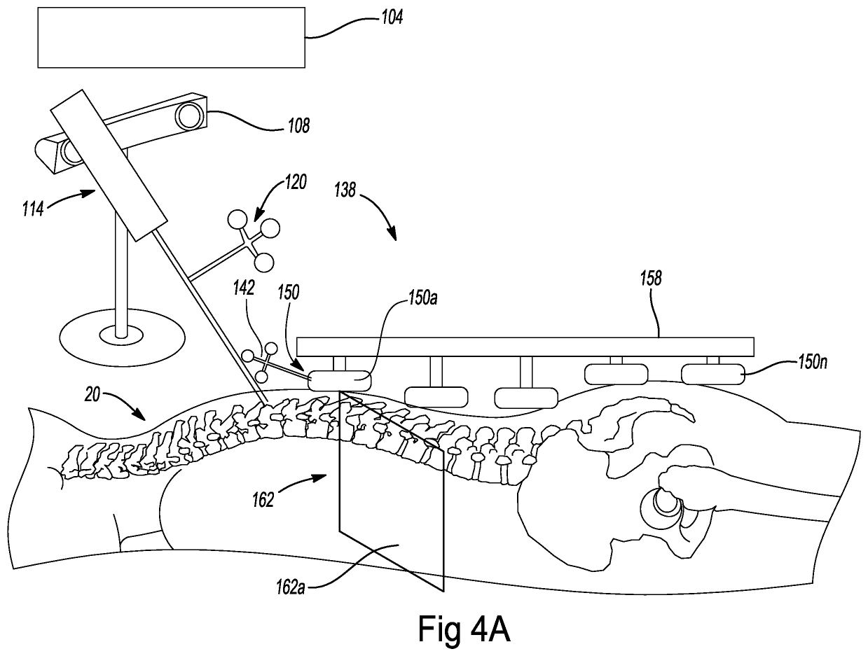

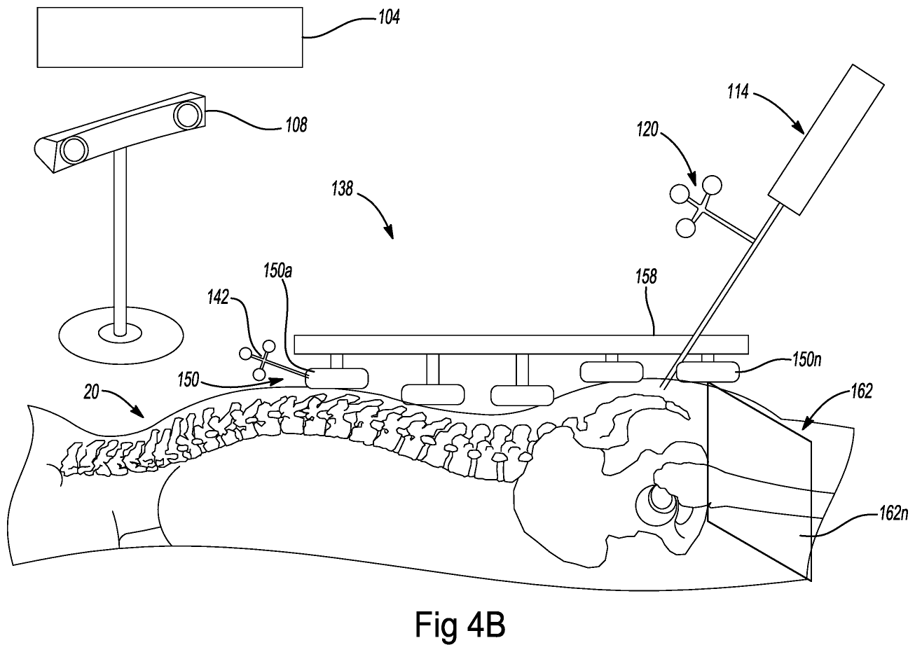

While manual switching may be performed between the ultrasound transducers 150, substantially automatic switching may also be performed. With reference to FIGS. and 4A and 4B, the respective ultrasound transducers 150 may be switched substantially automatically based on a process, as discussed further herein, to operate (e.g., on) and acquire image data with one of the respective transducers 150. As illustrated in FIG. 4A, the respective localizer, such as the EM localizer 104 and/or the optical localizer 108 may track the imaging system 138 and the device 114. As the device 114 is moved relative to the imaging system 138, a relative pose of the device 114 relative to the imaging system 138 may be determined.

For example, as the device 114 is moved relative to the first transducer 150a, the ultrasound transducer 150a may be operated to acquire image data in the transducer space or collect image data in the transducers space 162a. The US transducer 150a may be the only US transducer operating when the device 114 is determined to be near the US transducer 150a. The determination may be made based upon the tracked pose of the device 114 relative to the imaging system 138.

Turning reference to FIG. 4B, the device 114 may move relative to the transducer 150n. It is understood that the device 114 may be moved relative to any of the US transducers 150 and the US transducer 150n is merely exemplary. Nevertheless, the tracked pose of the device 114 may be used to determine its position relative to the US transducer 150n. The imaging system 138, therefore, may then turn off operation of the US transducer 150a and operate the US transducer 150n to acquire image data in the transducer space 160n. Thus, if the US transducer 150n may be operated substantially alone without operation of the US transducer 150a.

As discussed above, therefore, operation of the respective US transducers 150 may be performed substantially individually and separately to acquire image data of the subject 20 when the device 114 is determined to be at the selected relative pose relative to one or more of the respective US transducers 150 of the imaging system 138. Again, this may allow for operation of only one or less than all of the US transducers 150 based upon the determined tracked pose of the device 114 relative to the imaging system 138.

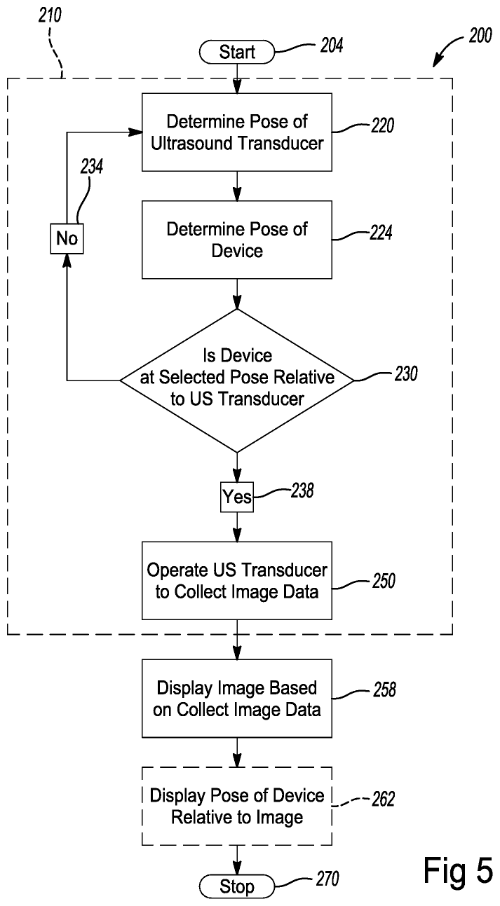

Accordingly, the imaging system 138 may be operated to acquire image data of the subject 20 based upon a determined pose of the device 114. The pose of the device 114 may include location and orientation information. Therefore, the pose of the device 114 may allow for a determination of an appropriate one or more of the ultrasound transducers 150 to be operated to acquire image data of an appropriate portion of the subject 20 to be displayed on the display device 62. As the image data is generally acquired in substantially real time, therefore, the user 52 may view a real time image of the subject 20 and of the instrument 114 relative to the portion of the subject 20. With continuing reference to FIGS. 4A and 4B and additional reference to FIG. 5, a method or process 200 is illustrated. The process 200 relates to FIGS. 4A and 4B and may be carried out by executing selected instructions on any one of the processing module's disclosed herein, including the processing module 50 of the navigation system and/or the imaging processor 170.

The process 200 may begin and start block 204. The process 200 may include a sub-process 210 including various steps or procedures rendered to assist in determining the pose of the device 114. A pose of the US transducer is performed block 220. A pose of the device 114 is determined in block 224. As noted above, the pose of the US transducer 150 and the device 114 may include various information for determining location and orientation. Further, the pose of the US transducer and device may occur in any appropriate order and the order illustrated in FIG. 5 is merely exemplary.

A determination of whether the device 114 is at selected pose relative to a US transducer 150 is made in block 230. A selected pose of the device relative to one or more of the transducers 150 may include a distance therefrom, orientation relative thereto, position relative to an imaged portion of the subject 20, or the like. For example, a selected relative pose may include that the device 114 or a selected portion thereof is less than 1 cm away from the plane defined relative to a selected one of the US transducers 150. Other appropriate selected relative poses of the device 114 may also be used. Nevertheless, if it is determined that the device 114 is not within a selected pose relative to any of the transducers 150, a NO path 234 may be followed. When following the NO path 234, the determination of the pose of the US transducer in block 220 and the pose of the device 114 in block 224 may be repeated. Therefore, the process 200 may allow for a loop process to continually update the determined poses of the US transducer 150 and the device 114.

If it is determined that the device is at a selected pose relative to the US transducer, a YES path 238 may be followed. After the YES path 238 is followed, the US transducer that is in the selected pose relative to the device is operated in block 250. Operating the US transducer includes acquiring image data with the selected US transducer, such as the first transducer 150a. As noted above, the imaging device 138 may include a plurality of the US transducers 150. Therefore, operation of the selected US transducer includes operation of the one US transducers 150 or appropriate number of US transducers 150 to acquire image data with the imaging system 138.

The acquired image data may be used to display an image, such as the image 110, on the display device 62 in block 258. The display of the image 110 is optional and may be displayed for the user 52 or may be for analysis this of the position of the device 114 relative to the subject 20. Similarly, a display of the pose of the device 114, such as with the graphical representation 114′, may be displayed in block 262. Again, the display of the pose the device on the display device and 62 is optional and need not be required.

The process 200 may then stop in block 270. Thus, the process 200 may allow for a determination of one or more of the use transducers 150 to be operated to acquire image data of the subject 20 based upon a determined relative pose of the device 114 to one or more of the US transducers 150. The acquired image data may then be selectively and/or optionally displayed on the display device 62 either alone or with a graphical representation of the device 114.

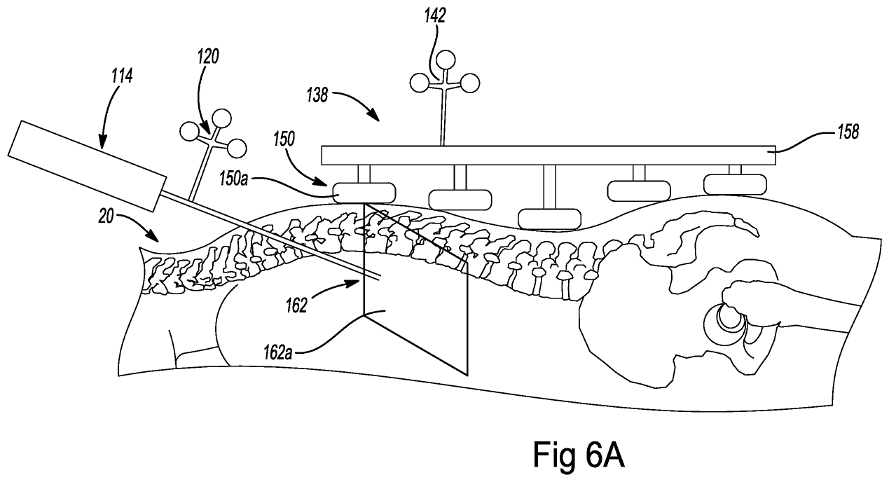

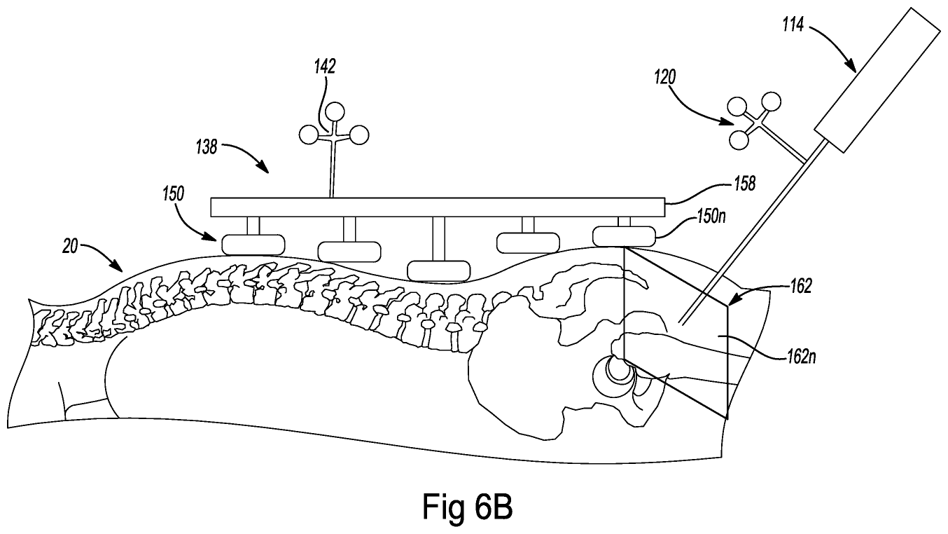

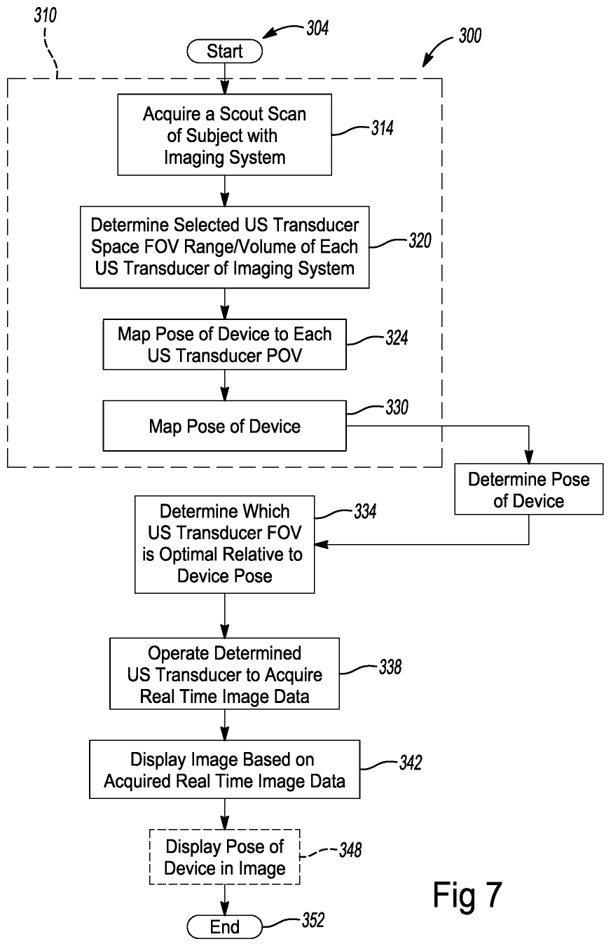

With continuing reference to FIGS. 1 through 3 and additionally to FIGS. 6A, 6B, and 7, the imaging system 138 may be operated to selectively image portions of the subject 20 substantially automatically and/or with minimally manual intervention. Again, the imaging system 138 may be positioned relative to subject 20. The device 114 may be moved relative to the subject 20 and also to the imaging system 138. As discussed above, the imaging system 138 includes a plurality of the US transducers 150. Each of the US transducers 150 may image an area or volume that they may also be referred to as the transducer plane or space 162. Each of the US transducers 150 generate image data in the area 162 and may include the device 114. Selected ones of the US transducers may be operated when the device 114 is moved within and/or a selected distance relative to the space 162.

For example, the imaging system 138 may be positioned relative to the subject 20. A scout or initial scan may be made with each of the ultrasound transducers 150 of the imaging system 138 to predetermine the transducer space 162 for each of the US transducers 150 relative to the subject 20. These may then be saved for later recall to determine which of the US transducers 150 may be operated to image the subject 20 when the device 114 is determined to be relative thereto. The scout scan may include sequentially operating each of the US transducers 150 and/or operating them in any appropriate manner to acquire an initial scan. The initial scan image in the volume 162 may then be used to determine an area of the subject 20 that may be imaged with each of the respective US transducers 150.

FIG. 7 illustrates a process 300 that may be used to determine which of the US transducers 150 to operate to image the subject 20 based at least upon a relative pose of the device 114, as illustrated in the system of FIGS. 6A and 6B. The process 300 may be the performed by executing instruction with one or more of the processing modules, such as the processor module 50. The process 300 may start in block 304. The process 300 may enter a sub-process 310. The sub-process 310 may operate to selectively determine which of the US transducers 150 to operate. In the sub-process 310, a scout scan may be made with the imaging system 138 in block 314. As noted above, the scout scan may initially acquire image data with each of the US transducers 150. Briefly, the scout scan acquired in block 314 allows for a determination of the volume or transducer space for each of the US transducers 150.

Once the scout scan is created, the transducer space 162 for each of the US transducers 150 is determined and a determination of a selected range or volume of each of the US transducers 150 of the imaging system 138 is made in block 320. Thus, the scout scan that acquires the scan of the subject 20 for each of the US transducers allows for analysis of the acquired image data to determine a selected volume or transducer space for each of the US transducers 150. The determination of the US transducer space 162 for each of the US transducers 150 allows for the transducer space 162 to be analyzed or have a range determined that is best or optimal for imaging the subject 20 when the device 114 is at a selected pose relative thereto. As noted above, the imaging system 138 may be tracked with the imaging system tracker 142 and/or each of the US transducers 150 may be tracked. Therefore, the transducer space 162 may also be tracked based upon a known volume or image space relative to the US transducers 150, such as that disclosed in U.S. Pat. No. 9,138,204 or U.S. Pat. No. 8,320,653, both incorporated herein by reference. The transducer space may also be referred to as a field of view (FOV) of each of the US transducers 150.

Once the transducer space or field of view is determined for each of the US transducers in block 320, a pose of the device 114 relative to the field of view for selecting a US transducer may be determined in block 324. As discussed above, real-time image data may be acquired of the subject 20 when the device 114 is at a selected position relative to the subject 20. As the real time image data may be acquired with a selected one of the US transducers 150, the selected FOV to acquire the image data most appropriate relative to the device 114 may be made in block 324. For example, the transducer space that is determined in block 320 may be used to determine a volume or patient space that is best imaged with a selected one of the US transducers 150. The device 114, therefore, when tracked relative to the subject 20 may be determined to be in a selected pose relative to the transducer space of a selected US transducer 150. The selected US transducer may be operated to acquire image data of the subject and/or the device 114 when at a predetermined mapped pose. The mapping may be substantially automatic based upon a volume of the FOV of the US transducer 150 based upon the determined FOV as noted above.

Once the sub-process 310 has determined the appropriate pose for operating in the selected US transducer 150 to acquire appropriate image data relative to the device 114, a pose of the device may be determined in block 330. Again, the determined transducer space or field the view 162 of the US transducer 150 may be determined in block 320 and may be tracked or registered relative to the subject 20, as is generally known in the art and as discussed above. Therefore, a determined pose appropriate to operate one or more of the US transducers 150 to appropriately image or selectively image the subject 20 relative to the tracked device 114 may be known based upon a tracked pose of the device 114 relative to the subject 20. During use, in determining a pose of the device in block 330 may be determining of a pose of the device 114 relative to any of the field of view of the transducers 150. The determination of which of the US transducer field of view is appropriate for imaging the subject and/or within the pose of the device 114 may be determined in block 334

Once the determined US transducer FOV is made in block 334, the determined US transducer may be operated to acquire real time image data in block 338. As noted above, once the device 114 is determined to be within the field view of a selected one of the US transducers 150, that US transducer may be operated to acquire image data of the subject 20. The image data acquired with the selected US transducer 150 may be substantially real time image data and may be displayed in block 342, if selected.

Image data may be displayed on the display device 62 as the image 110. Optionally, a display of the determined the pose of the device 114 may be displayed as the graphical representation 114′ in block 348.

The process 300 may then end in block 352. Thus, the process 300 may allow for a substantially automatic selection of the US transducer 150 that is best or optimally positioned to image relative to the device 114. Again, the optimal position may be based upon a selection of the user 52 based on a determined pose of the device 114 relative to the subject 20 and/or one or more of the US transducers 150 or other appropriate determination. The process 300 may allow for the transducer space 162 of only a selected one of the US transducers to be used to acquire image data at a given time to eliminate an interference and/or other difficulties of operating a plurality of the US transducers simultaneously close to one another and/or close of the subject 20. Further, the imaging device or system 138 allows for the multiplexing or switching to be substantially automatic and not require movements of the imaging system 138 during the procedure.

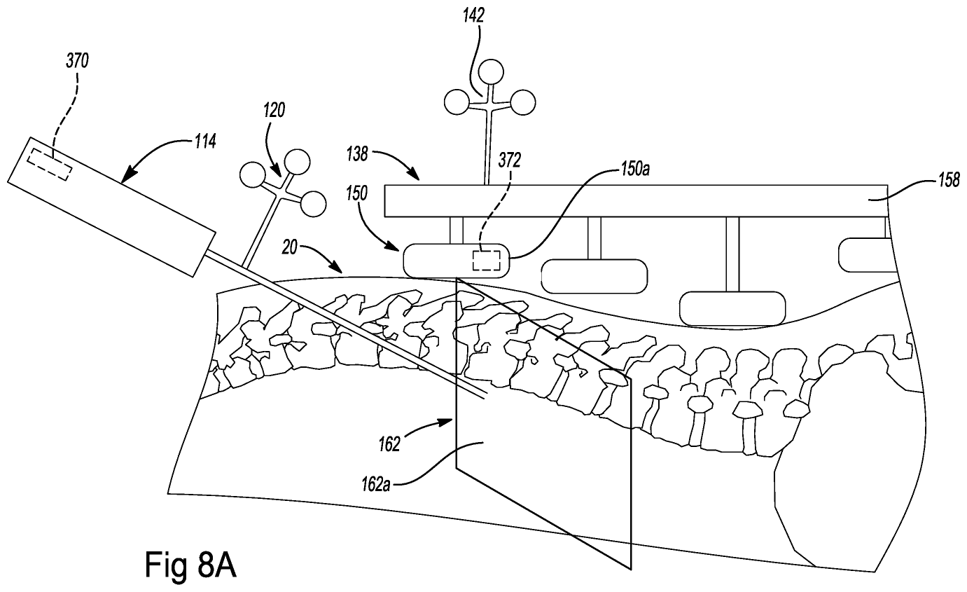

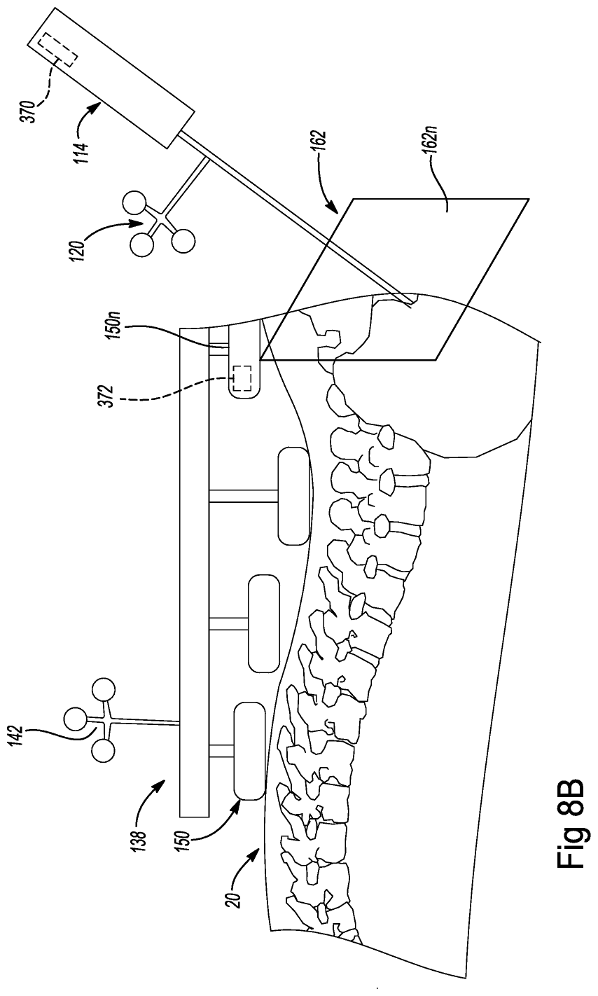

With continuing reference to FIGS. 1 through 3 and additional reference to FIGS. 8A and 8B, the imaging system 138 may be operated to selectively operate one or more of the US transducers 150 substantially individually and/or in a selected smaller group to image the subject 20 relative to the device 114. As discussed above, the imaging system 138 may be tracked and the transducer space of each of the US transducers 150 may be registered relative to the subject 20. Each of the US transducers 150 acquire image data within the respective transducer spaces 162.

The US transducers 150 may sense the device 114 in an appropriate manner. For example, a pulsed image or selected imaging pulses may be made with all of the US transducers 150 at a selected rate. For example, each of the US transducers 150 may sequentially or in an appropriate order image the subject 20 at a selected rate, such as once every second, ever 5 seconds, every 30 seconds, or any appropriate rate. Generally, a rate may include once every 0.5 seconds to about once every 30 seconds. The image data acquired with each of the US transducers 150 during the pulse image data collection may be used to determine the position of a selected portion of the device 114. If the selected portion of the device 114 is sensed within the transducer space of a selected one or more of the transducers, that transducer may be operated to generate image data of the subject. Similarly, the image data may be acquired and analyzed to determine that the device 114 has left the transducer space of a selected one of the transducers. Adjacent transducers may be operated to determine the position of the device 114 and thereafter generate image data of the subject 22 image the subject 20 relative to the device 114. In other words, the collection of image data may be handed off from one of the US transducers to another.

Other sensors may also be provided, such as a proximity sensor, material sensor, or the like. For example, the device 114 may include a selected sensing portion, such as a radio frequency (RF) transmitter. The imaging system 138 may include a receiver to sense of the position of device 114 relative to a selected one of the US transducers based upon the sensed portion. A sensed portion may include a RF tag 370 that may be sensed by or relative to one or more of the US transducers 150 on the imaging system 138. For example, a sensor 372 may be included with each of the US transducers 150 to sense proximity of the sensor portion 370. Again, the appropriate US transducer may then be used to generate image data of the subject 20 based upon the sense position of the device 114.

In a similar manner the tracking systems may sense a proximate pose of the US transducers, including a specific one of the US transducers 150 and the device 114. As noted above, the tracking systems may track the device tracking device 120 to determine the pose of the device 114. The pose of the US transducers 150 may also be determined. In both cases, the respective tracking devices 142, 120 are sensed. An appropriate processor, such as the navigation system processor 50 may calculate or determine that the pose of the device 114 is within a FOV of at least one of the US transducers 150 and the US transducer may be operated to acquire image data including the device 114.

Other selected sensing mechanisms may also be used to sense the position of the device 114 relative to the US transducer to be operated to acquire image data of the subject 20. Nevertheless, of the sensing of the device 114 may allow a selected one of the US transducers to be operated substantially independently or alone to generate the image data of the subject 20 relative to the device 114. Thus, the image data may be acquired without interference of operation of the other US transducers, as discussed above.

According to various embodiments, including those noted above, the image data acquired with the imaging system 138 may be used to generate the image 110 displayed on the display 62. The image may be generated based upon a plurality of the image data acquired with a plurality of the US transducers 150 of the imaging system 138. The plurality of image data from the plurality of US transducers 150 may be analyzed to generate a long image or a stitched image of the subject 120. The stitched image data may be stitched in any appropriate manner, such as those understood by one skilled in the art. The stitching may be based on detecting at least one anatomical landmark in multiple images, such as based on the position of the many US transducers 150 that produce such images. The at least one anatomical landmark may be the same landmark identified in each image from each of the respective transducers 150. Identification methods may include a machine learning algorithm as described herein and/or based on matching a pre-operative images and intraoperative image segments.

In various embodiments features within the image data may be identified. The identified features may be based upon a selected algorithms and/or machine learning systems. For example, a learning aleatoric algorithm may be used to identify various thresholds and/or features in the image. Similarly various machine learning systems may include artificial intelligence or neural networks to identify features in the image data. The selected machine learning systems may be trained with acquired image data to assist in identifying features in the image data acquired with the imaging system 138. These systems may be trained to automatically identify selected features, e.g., spinous process or fiducials, in respective images to allow for stitching of multiple images.

As noted above, the imaging system 138 may be tracked with the image tracker 142. Similarly, the subject 20 may be tracked with the subject tracker 100. Thus, the image data acquired with the imaging system 138 may be registered to the subject 20. Further, the simultaneous tracking of the subject 20 and of the imaging system 138 may allow for maintenance of the registration during a procedure, even if one or more of the subject and/or the imaging system 138 removed. As is understood by one skilled in the art, pre-acquired image data may be registered to the real time image data or other image data acquired with the imaging system 138 to assist in various procedures. For example, computed tomography and/or magnetic resonance imaging image data may be registered to image data acquired with the imaging system 138. The registered image data may also be displayed with the display device 62 and/or any appropriate imaging device to display information from the pre-acquired or alternatively acquired image data.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

Instructions may be executed by a processor and may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The apparatuses and methods described in this application may be partially or fully implemented by a processor (also referred to as a processor module) that may include a special purpose computer (i.e., created by configuring a processor) and/or a general purpose computer to execute one or more particular functions embodied in computer programs. The computer programs include processor-executable instructions that are stored on at least one non-transitory, tangible computer-readable medium. The computer programs may also include or rely on stored data. The computer programs may include a basic input/output system (BIOS) that interacts with hardware of the special purpose computer, device drivers that interact with particular devices of the special purpose computer, one or more operating systems, user applications, background services and applications, etc.

The computer programs may include: (i) assembly code; (ii) object code generated from source code by a compiler; (iii) source code for execution by an interpreter; (iv) source code for compilation and execution by a just-in-time compiler, (v) descriptive text for parsing, such as HTML (hypertext markup language) or XML (extensible markup language), etc. As examples only, source code may be written in C, C++, C#, Objective-C, Haskell, Go, SQL, Lisp, Java®, ASP, Perl, Javascript®, HTML5, Ada, ASP (active server pages), Perl, Scala, Erlang, Ruby, Flash®, Visual Basic®, Lua, or Python®.

Communications may include wireless communications described in the present disclosure can be conducted in full or partial compliance with IEEE standard 802.11-2012, IEEE standard 802.16-2009, and/or IEEE standard 802.20-2008. In various implementations, IEEE 802.11-2012 may be supplemented by draft IEEE standard 802.11ac, draft IEEE standard 802.11ad, and/or draft IEEE standard 802.11ah.

A processor, processor module, module or ‘controller’ may be used interchangeably herein (unless specifically noted otherwise) and each may be replaced with the term ‘circuit.’ Any of these terms may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the invention, and all such modifications are intended to be included within the scope of the invention.

What is claimed is:

1. A method of imaging a subject, comprising:
providing a plurality of ultrasound transducers relative to a subject;
determining a pose of a device relative to at least one of the subject or the plurality of ultrasound transducers;
determining at least one ultrasound transducer of the plurality of ultrasound transducers for acquiring image data of the subject based at least on the determined pose of the device relative to at least one of the subject or the plurality of ultrasound transducers; and
acquiring image data with the determined at least one ultrasound transducer.

2. The method of claim 1, further comprising:
providing the plurality of ultrasound transduces relative to the subject.

3. The method of claim 2, further comprising:
providing the plurality of ultrasound transducers fixed relative to each other.

4. The method of claim 1, wherein acquiring image data with the determined at least one ultrasound transducer includes the acquiring real time image data.

5. The method of claim 1, further comprising:
multiplexing the plurality of ultrasound transducers to acquire the image data with the determined at least one ultrasound transducer.

6. The method of claim 5, wherein the multiplexing includes at least manually operating the determined at least one ultrasound transducer.

7. The method of claim 6, further comprising:
operating a switch to operate the determined at least one ultrasound transducer.

8. The method of claim 5, wherein the multiplexing comprises:
tracking the device to determine the pose of the device;
determining that a selected portion of the device is within a selected proximity of the at least one of the ultrasound transducers of the plurality of ultrasound transducers; and
operating the at least one ultrasound transducer to acquire the image data.

9. The method of claim 5, wherein the multiplexing comprises:
generating an initial scan of the subject with the plurality of ultrasound transducers;
determining a field of view of each ultrasound transducers of the plurality of ultrasound transducers;
tracking the device to determine the pose of the device;
determining the determined field of view that a selected portion of the device is within or within a selected proximity of; and
operating the ultrasound transducer having the determined field of view to acquire the image data.

10. The method of claim 5, wherein the multiplexing comprises:
sensing a proximity of the device relative to at least one of the ultrasound transducers of the plurality of ultrasound transducers; and
operating the at least one ultrasound transducer to acquire the image data.

11. A system to image a subject, comprising:
a plurality of ultrasound transducers configured to be positioned relative to a subject;
a device configured to be positioned relative to at least one of the subject or the plurality of ultrasound transducers;
a multiplexer configured to operate at least one ultrasound transducer of the plurality of ultrasound transducers for acquiring image data of the subject based at least on a pose of the device relative to at least one of the subject or the plurality of ultrasound transducers; and
acquiring image data with the determined at least one ultrasound transducer.

12. The system of claim 11, further comprising:
a tracking system comprising a device tracker and at least one ultrasound transducer tracker;
wherein the tracking system is configured to determine the pose of the device relative to the at least one ultrasound transducer.

13. The system of claim 11, wherein the multiplexer includes a manual switch configured to allow manually operating the determined at least one ultrasound transducer.

14. The system of claim 13, wherein the manual switch includes an electronic switch that is manually selected by a user.

15. The system of claim 12, further comprising:
a processor module configured to execute instructions to:
determine the pose of the device based at least on tracking the device tracker; and
wherein the multiplexer is configured to execute instructions to:
determine that a selected portion of the device is within a selected proximity of the at least one of the ultrasound transducers of the plurality of ultrasound transducers; and
operate the at least one ultrasound transducer to acquire the image data.

16. The system of claim 12, further comprising:
a processor module configured to execute instructions to:
evaluate an initial scan of the subject with the plurality of ultrasound transducers to determine a field of view of each ultrasound transducers of the plurality of ultrasound transducers;
determine the determined field of view that a selected portion of the device is within or within a selected proximity of based at least on the determined pose of the device; and
wherein the multiplexer is configured to execute instructions to operate the ultrasound transducer having the determined field of view to acquire the image data.

17. The system of claim 11, further comprising:
a proximity sensor included with at least one of the ultrasound transducers of the plurality of ultrasound transducers;
a sense portion included with the device;
wherein the proximity sensor is configured to sense the sense portion to determine a proximity of the device relative to at least one of the ultrasound transducers of the plurality of ultrasound transducers; and
wherein the multiplexer is configured to execute instructions to operate the at least one ultrasound transducer to acquire the image data.

18. A system to image a subject, comprising:
an imaging system comprising:
a placement member;
a plurality of ultrasound transducers configured to be positioned relative to a subject; and
a multiplexer configured to selectively operate one ultrasound transducer of the plurality of ultrasound transducers for acquiring image data of the subject based at least on a pose of the device relative to at least one of the subject or the plurality of ultrasound transducers;
wherein the operated one ultrasound transducer is configured to acquire image data.

19. The system of claim 18, further comprising:
a display device to display an image based on the acquired image data.

20. The system of claim 18, further comprising:
a device configured to be positioned relative to at least one of the subject or the plurality of ultrasound transducers;
wherein the multiplexer selectively operates the one ultrasound transducer based on the position of the device.
